(19) United States
(12) Patent Application Publication
(10) Pub. No.: US 2017/0209204 A1
(43) Pub. Date: Jul. 27, 2017

(54) ENDOSCOPIC SURGICAL INSTRUMENT

(71) Applicant: KUAN-YU LIN, KAOHSIUNG CITY (TW)

(72) Inventor: KUAN-YU LIN, KAOHSIUNG CITY (TW)

(21) Appl. No.: 15/007,497

(22) Filed: Jan. 27, 2016

(51) Int. Cl. A61B 18/14; A61B 17/32

(52) U.S. Cl. CPC A61B 18/148; A61B 17/32002; A61B 2018/00595

(57) ABSTRACT

An endoscopic surgical instrument is revealed herein to comprise a handle, a hollow tube fitted at a front end of the handle and having an oblique opening at a distal end thereof, a repair device fitted inside the hollow tube and having a cutting portion partially exposed to the oblique opening, and an electrocautery device fitted on an end surface of the oblique opening of the hollow tube.

### BACKGROUND OF THE INVENTION

Field of the Invention

The present invention relates to an endoscopic surgical instrument, particularly to an endoscopic instrument that served to both remove soft tissue and stop bleeding in articular joints during surgical operations.

Description of Related Art

An articular joint is an area of the body where two bones are connected for the purpose of permitting body parts to move. It is enclosed by an articular capsule, which is fibrous in nature, and is lined by synovial membrane. The synovial membrane lines the entire joint except the articular surfaces. It is this membrane that secretes the viscous fluid called synovial fluid, which lubricates the joint and nourishes the articular cartilage. The articular surfaces are covered with hyaline cartilage, which provides a smooth, lubricated surface for articulation and facilitates the transmission of loads with a low frictional coefficient. However, because articular cartilage is devoid of blood vessels, lymphatics, and nerves and is constantly subjected to a harsh biomechanical environment, it has a limited capacity for intrinsic healing and repair when injured or worn. Injury or degeneration of articular cartilage is thus recognized as a cause of significant musculoskeletal morbidity. When the synovial joint is inflamed and becomes painful and swollen, it is termed arthritis, which eventually results in an irreversible destruction of the articular cartilage and severe hindrance of daily activity. According to the survey by the Arthritis Foundation, more than 50 million adults and 300,000 children have some type of arthritis in the US. Two of the most common types of arthritis are osteoarthritis (OA) and rheumatoid arthritis (RA) with the former affects mainly the elderly while the latter could occur in any age group.

Treatment of arthritis focuses mainly on relieving symptoms and improving joint function, and is generally divided into non-operative and operative. Non-operative modalities include medication, such as nonsteroidal anti-inflammatory drugs (NSAIDs), disease-modifying anti-rheumatic drugs (DMARDs for RA), and corticosteroids, as well as physical therapies. When arthritic symptoms are unresponsive to aforementioned conservative treatments, surgical measures such as arthroscopy or total joint replacement may be considered. While total joint replacement is reserved for the severely deformed joints, those with less degenerative or destructed may be managed with less invasive arthroscopic procedures. For joints with seriously damage, a surgical operation could be considered, such as arthroscopic procedures or total joint arthroplasty. The surgical operation is a treatment for the patient suffering from joint deformity, unstable joint, or damaged joint. Some of the most commonly surgical procedures are endoscopic (arthroscopic) synovectomy, cartilage repair, tendon or ligament repair or reconstruction, and osteotomy.

Generally speaking, there are many different types of endoscopes used in surgical operations, depending on sites in a human body and types of procedures. Endoscopy is usually performed by a doctor or a surgeon with suitable endoscopes for examination or treatment of internal organs or cavities, and patients to be examined or treated may be fully or partially conscious or anaesthetized.

For conventional endoscopic surgical instruments, U.S. Pat. No. 5,904,681 discloses surgical instruments which have multiple functions used in performing endoscopic surgeries, including arthroscopic surgery. It shows a surgical instrument 10, as shown in FIGS. 3 and 6, to comprise a probe 92 having an opening at a distal end 96 thereof, a rotatable shaft 102 longitudinally disposed within the probe 92, and a serrated blade 104 connected to the shaft 102. The distal end 96 of the probe 92 also includes a bipolar device assembly 134. Damaged tissues can be removed by the serrated blade 104 of the shaft 102, and then bleeding tissues are ablated and/or cauterized. However, the foregoing invention has the following disadvantages.

1. The bipolar device fitted at a position on the opening of the distal end opposite to the rotating burr needs the probe having a larger diameter, so that the surgical instrument will be subject to a limited flexibility of use due to a small joint cavity.

2. When the synovium or damaged tissues are removed by the serrated blade of the shaft, bleeding will easily occur due to dense distribution of blood vessels to further result in an unclear surgical visual field. Additionally, the probe needs to be turned in operation because the bipolar device fitted at a position on the opening of the distal end opposite to the rotating burr, and the time spent for turning the probe will easily cause more bleeding to unfavorably affect surgical operations.

### SUMMARY OF THE INVENTION

Therefore, the object of the present invention is related to an endoscopic surgical instrument that works to remove intended soft tissue and stop in-bedded vessel bleeding either independently or simultaneously, thus maximizing the clarity of operating field. This invention is designed for purposes of ensuring surgical safety and shortening operation time.

For the above object, an endoscopic surgical instrument comprises a handle, a hollow tube fitted on a front end of the handle, a repair device fitted inside the hollow tube and an electrocautery device. The hollow tube has an oblique opening at a distal end thereof The repair device has a cutting portion at an end thereof partially exposed to the oblique opening of the hollow tube for cutting damaged joint tissues. The electrocautery device is fitted at a certain position in a direction of the oblique opening of the outer tube. During an arthroscopic procedure, the endoscopic surgical instrument of the present invention is used to cauterize the wound in a real-time manner as the damaged joint tissues are immediately cut, for rapidly stopping bleeding and allowing surgery operations to be performed without obstacles.

According to an embodiment of the present invention, the certain position where the electrocautery device is fitted is preferably right on the oblique opening, for real-time treatment of surgical sites.

According to an embodiment of the present invention, the endoscopic surgical instrument comprises a first button on the handle for controlling and actuating the repair device.

According to an embodiment of the present invention, the endoscopic surgical instrument comprises a second button on the handle for controlling and actuating the electrocautery device.

According to an embodiment of the present invention, the endoscopic surgical instrument comprises a third button on the handle for simultaneously controlling and actuating the repair device and the electrocautery device.

According to the above description and embodiments, the endoscopic surgical instrument of the present invention enables to cauterize the wound simultaneously with burring or shaving, thus rapidly stop the wound from bleeding, and keep a clear visual field of the arthroscopic procedure without unfavorably influencing healthcare providers' determination and judgment in ensuring surgical safety. These advantages are aided by the design of the electrocautery device and the cutting portion of the present invention being in close proximity to each other in the same direction.

### BRIEF DESCRIPTION OF THE DRAWINGS

FIG. 1 is a three-dimensional view of an endoscopic surgical instrument according to a first embodiment of the present invention;

FIG. 2 is a three-dimensional exploded view of an endoscopic surgical instrument according to a first embodiment of the present invention;

FIG. 3 is a perspective view of an endoscopic surgical instrument in assembly according to a first embodiment of the present invention;

FIG. 4 is a partial enlarged cross-sectional view of an endoscopic surgical instrument according to a first embodiment of the present invention;

FIG. 5 is a perspective view of an endoscopic surgical instrument in assembly according to a second embodiment of the present invention; and

FIG. 6 is a perspective view of an endoscopic surgical instrument in assembly according to a third embodiment of the present invention.

### DETAILED DESCRIPTION OF THE PREFERRED EMBODIMENT

First, arthroscopy is a specific type of endoscopy. Taking the knee arthroscopy for example, it is performed to treat diseases indicating meniscus and arthrodial cartilage damages, ligament injuries or tear, osteochondral or cartilage fractures, floating loose bodies of knee joints, arthritis and synovitis, synovial fold thickening, patellofemoral pain syndrome, joint infections, etc. All syndromes, such as joint disorders, hematoma, chronic edema, movement limits and pain, caused by the pathological changes on the knee joint, can also be treated by the knee arthroscopy as well.

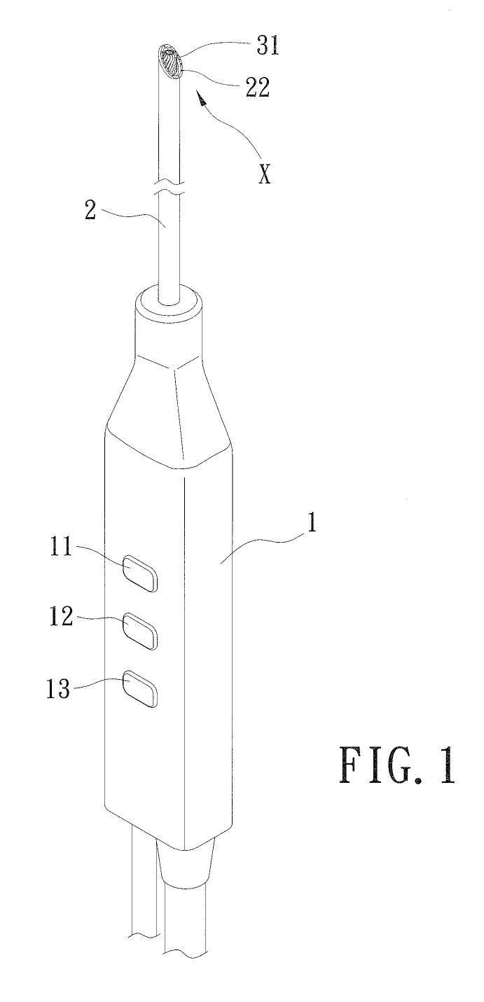

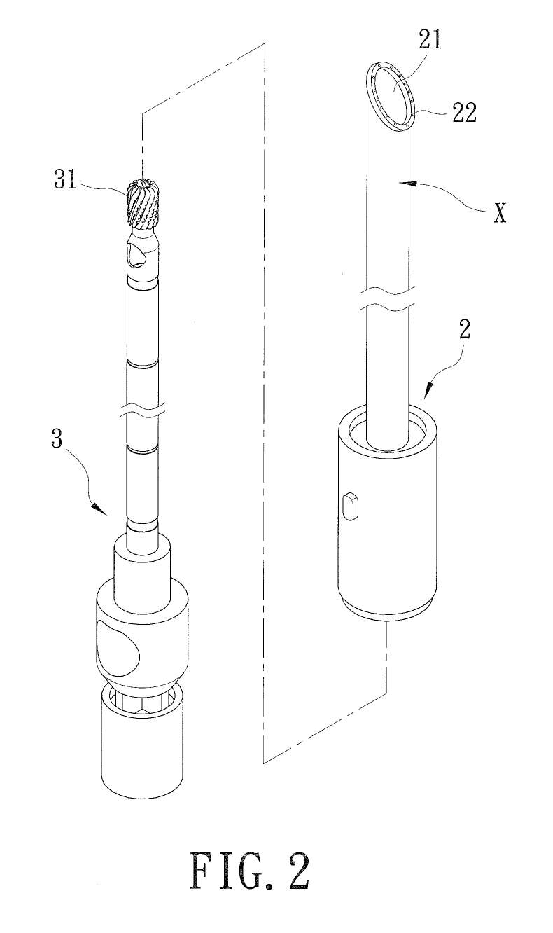

As referring to FIGS. 1 and 2 of an endoscopic surgical instrument according to a first embodiment of the present invention, it comprises a handle 1 for a user to control and manipulate an arthroscope, a hollow tube 2 fitted on a front end of the handle 1, a repair device 3 fitted inside the hollow tube 2, and an electrocautery device for cauterizing wounds, wherein the hollow tube 2 has an oblique opening 21 formed on a distal end thereof, and the electrocautery device 22 fitted at a position X of an outer wall thereof, corresponding to the direction of the oblique opening 21, the position X preferably right on an end surface of the oblique opening 21, and wherein the repair device 3 has a cutting portion 31 on an end thereof partially exposed to the oblique opening 21 of the outer tube 2 for properly cutting damaged joint tissues.

In actual application, the endoscopic surgical instrument of the present invention is used in surgical procedures for treatments of joint diseases. Human joint is a closed structure as it is well protected from foreign object penetration directly. Therefore, during an arthroscopic procedure, small skin incisions (less than 1 cm) are made to allow the insertion of an arthroscope and its associated instruments, such as the endoscopic surgical instrument of the present invention, for the examination and possible treatments of the underlying joint diseases.

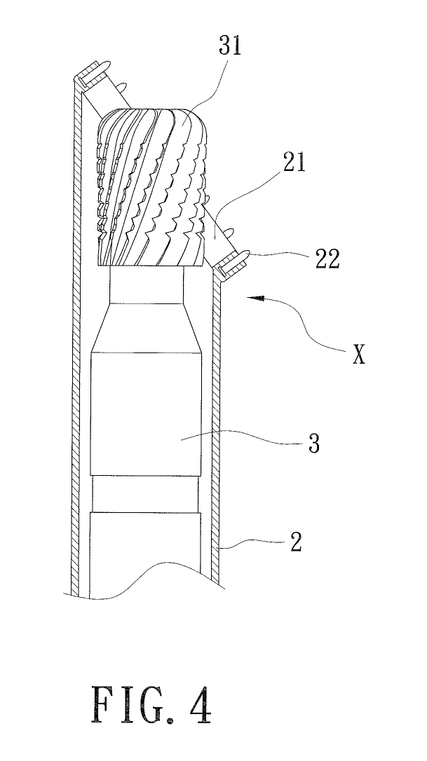

The surgical repair process after synovium is cut and removed is further described below, as also referred to FIGS. 1 and 4. It is noteworthy that the handle 1 of the present invention is further provided with a first button 11 for controlling and actuating the repair device 3, a second button 12 for controlling and actuating the electrocautery device 22, and a third button 13 for simultaneously controlling and actuating the repair device 3 and the electrocautery device 22. When an arthroscopic procedure is performed on a patient, the endoscopic surgical instrument is necessarily inserted into the joint as aforementioned, and a vacuum suction device (not shown) connected to a rear end of the endoscopic surgical instrument enables to attract the synovium at a disease site to move towards the cutting portion 31 of the repair device 3. Then, the first button 11 of the handle 1 is actuated to make the repair device 3 operate, and the synovial tissues inside the joint is cut and removed by rotation of the cutting portion 31 exposed to the oblique opening 21 of the hollow tube 2 while the removed tissues and blood from the wound are sucked out by the vacuum suction device in the course of cutting. Next, the second button 12 of the handle 1 is actuated to make the electrocautery device 22 fitted at a position X on an outer wall of the hollow tube 2, corresponding to the direction of the oblique opening 21 of the hollow tube 2, to cauterize the wound directly to stop the wound from bleeding. The electrocautery device 22 is preferably fitted right on an end surface of the oblique opening 21. In such a case, the surgery can be performed and completed without any obstacles, e.g. excessive bleeding from wounds to result in unclear visual field of surgery, and the patients' safety can be ensured during the surgery as well.

During endoscopic operations, healthcare providers can decide to perform both the cutting and cauterizing procedures by pushing the third button 13 to simultaneously control and actuate the repair device 3 and the electrocautery device 22, or pushing the first button 11 and the second button 12 in sequence as foregoing described, or solely pushing the second button 12 to cauterize the bleeding vessels or to vaporize the soft tissue at their discretion.

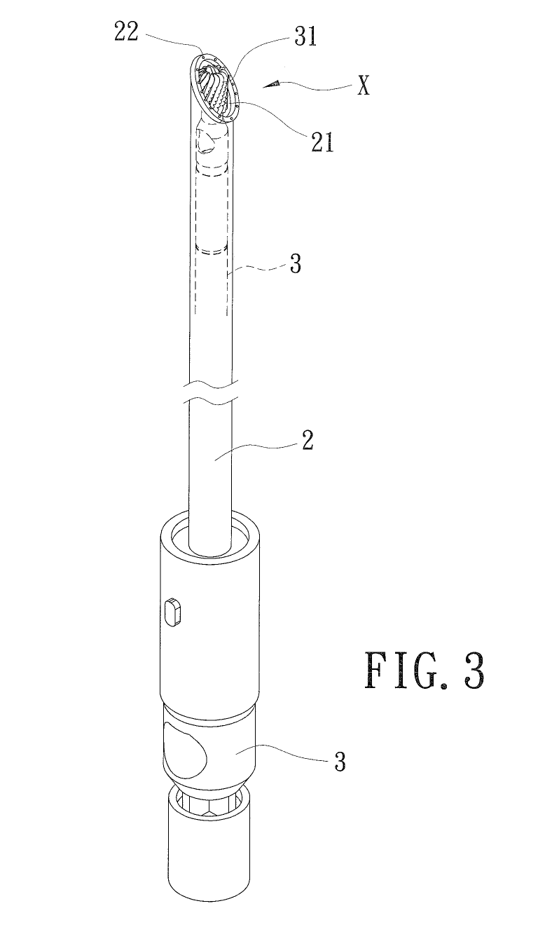

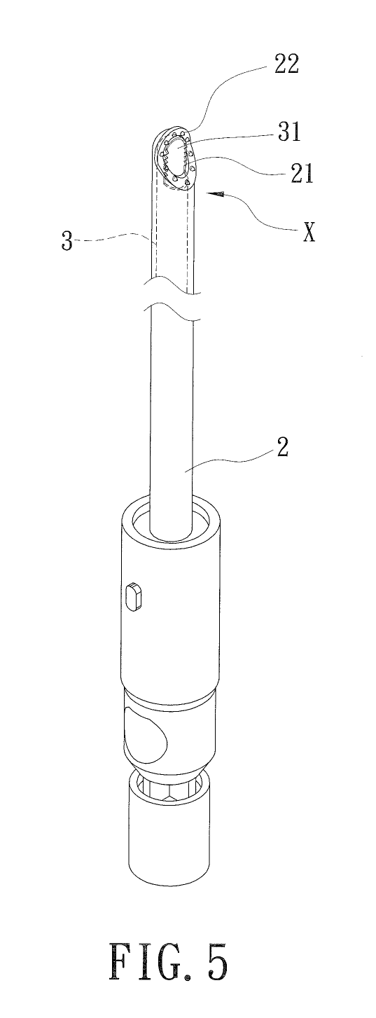

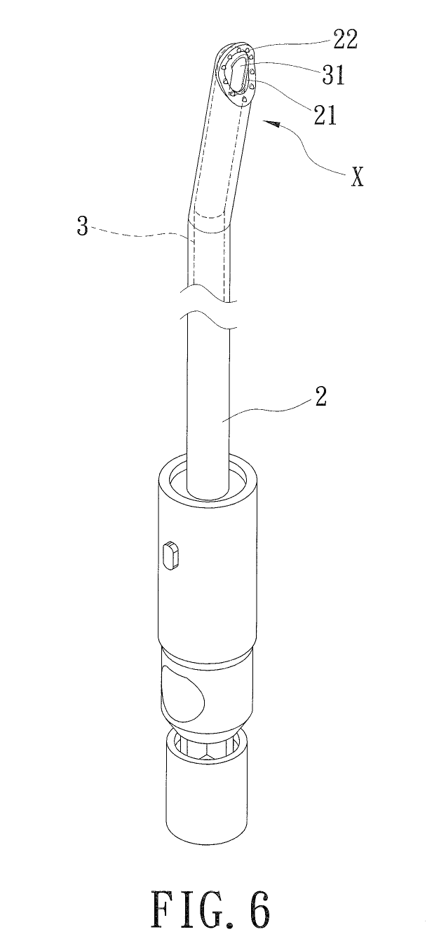

As further referring to FIG. 3, FIGS. 5 and 6, the endoscopic surgical instrument of the present invention has various outer configurations to be used in various surgical procedures as needed. According to a first embodiment of the present invention in FIG. 3, the hollow tube 2 is a straight tube and the cutting portion 31 of the repair device 3 is formed of serrated blades. According to a second embodiment of the present invention in FIG. 5, the hollow tube 2 is a straight tube, the oblique opening 21 has a curved cross section, and the cutting portion 31 of the repair device 3 is serrated at both sides thereof According to a third embodiment of the present invention in FIG. 6, the hollow tube 2 is a bent tube, the oblique opening 21 has a curved cross section, and the cutting portion 31 of the repair device 3 has a spatula-like shape. However, the foregoing embodiments and drawings does not limit the product structures or use of the present invention, it will be obvious to those skilled in the art that various modifications may be made without departing from the spirit and the scope of the present invention.

According to the above description and embodiments, the endoscopic surgical instrument of the present invention enables cauterization simultaneously with burring or shaving, thus rapidly stop the wound from bleeding, prevent the wound from infections and keep a clear visual field of the arthroscopic procedure without unfavorably influencing healthcare providers' determination and judgment for ensuring surgical safety. These advantages are aided by the design of the electrocautery device 22 and the cutting portion 31 of the present invention being in close proximity to each other in a same direction.

What is claimed is:

1. An endoscopic surgical instrument, comprising:
a handle for controlling and manipulating an arthroscope;
a hollow tube, fitted at a front end of the handle and having an oblique opening at a distal end thereof;
a repair device, fitted inside the hollow tube and having a cutting portion at an end thereof, wherein the cutting portion is partially exposed to the oblique opening of the hollow tube; and
an electrocautery device for cauterizing a wound, fitted at a position X on an outer wall of the hollow tube, corresponding to the direction of the oblique opening of the hollow tube.

2. The endoscopic surgical instrument as claim 1, wherein the electrocautery device is fitted right on an end surface of the hollow tube.

3. The endoscopic surgical instrument as claim 1, further comprises a first button fitted on the handle for controlling and actuating the repair device.

4. The endoscopic surgical instrument as claim 1, further comprises a second button fitted on the handle for controlling and actuating the electrocautery device.

5. The endoscopic surgical instrument as claim 1, further comprises a third button fitted on the handle for simultaneously controlling and actuating the repair device and the electrocautery device.
